(19) United States
(12) Patent Application Publication
(10) Pub. No.: US 2018/0104471 A1
(43) Pub. Date: Apr. 19, 2018

(54) MAGNETIC TRAPS GENERATED WITH PULSED MAGNETIC FIELDS FOR TARGETED DELIVERY

(71) Applicant: Advanced Magnet Lab, Inc., Palm Bay, FL (US)

(72) Inventors: Rainer Meinke, Melbourne, FL (US); Gerald Stelzer, Palm Bay, FL (US)

(73) Assignee: Advanced Magnet Lab, Inc., Palm Bay, FL (US)

(21) Appl. No.: 15/788,625

(22) Filed: Oct. 19, 2017

Related U.S. Application Data
(60) Provisional application No. 62/409,939, filed on Oct. 19, 2016.

(51) Int. Cl. A61N 1/04; A61M 31/00; A61M 37/00

(52) U.S. Cl. CPC A61N 1/0448; A61M 2205/0272; A61M 37/00; A61M 31/00

(57) ABSTRACT

A system and method for motivating a particle, for example a drug molecule, to a predetermined location in three dimensional space by applying magnetic fields, which may be static or time-varying, to the particle. The magnetic fields may be applied by one or a plurality of magnets, and may be multipole of any order such as octopole or decapole. The electric current driving the magnet coils may be pulsed for inducing a voltage in the molecules to aid in motivation. In an exemplary embodiment of the invention in which the substance is a drug, for example a drug molecule or plurality of drug molecules, the magnets may be positioned outside the body of the person to be treated, and the magnetic field(s), which may be time-varying, are used to motivate the drug molecule(s) to a predetermined location in an animal's body. The animal may be a human.

### CROSS REFERENCE TO RELATED APPLICATIONS AND INCORPORATION BY REFERENCE

This non-provisional application for patent is a non-provisional of U.S. provisional application Ser. No. 62/409,939 filed in the United States Patent and Trademark Office (USPTO) on Oct. 19, 2016, titled MAGNETIC TRAPS GENERATED WITH PULSED MAGNETIC FIELDS FOR TARGETED DELIVERY, which is hereby incorporated by reference in its entirety.

### STATEMENT REGARDING FEDERALLY SPONSORED RESEARCH OR DEVELOPMENT

Not applicable.

### INCORPORATION-BY-REFERENCE OF MATERIAL SUBMITTED ON A COMPACT DISK

Not applicable.

### BACKGROUND OF THE INVENTION

### 1. Field of the Invention

The invention pertains generally to the use of magnetic traps, or regions of space in which a magnetic field, or a plurality of superimposed magnetic fields, form a null region, or potential well region, that may be manipulated in three dimensional space. Such regions are useful for motivating particles to a predetermined location without the need for physical manipulation of the particle. The field of the invention includes, in one embodiment, systems and methods for particle manipulation. In an exemplary embodiment, the invention also applies to the field of deliver of drugs to specific locations or regions in the body of an animal such as a human patient. The invention is useful for delivering a substance to predetermined point in space, or to motivate, or in other words navigate, a substance along a predetermined path in space. In one example, the substance may be a drug molecule or plurality of molecules, and the invention may be adapted to motivate a drug molecule or plurality of molecules to a predetermined point in space, or to motivate, or in other words navigate, a drug molecule or plurality of molecules along a predetermined path in space.

### 2. Background Art

In traditional drug delivery such as oral ingestion or intravascular injection, the medication is distributed throughout the whole body via the systemic blood circulation system and, as a result, only a small portion of the medication reaches the organ or area of the body, to be affected. Targeted drug delivery seeks to concentrate the medication in the tissues of interest, while reducing the relative concentration of the medication in the remaining tissues. By avoiding defense mechanisms of the body and inhibiting drug distribution in the liver and spleen, a drug can reach the intended site of action in higher concentrations, thereby improving efficacy of the drug and reducing side-effects.

What is needed in the art, therefore, is a system or method, or both, for motivating a drug molecule or plurality of drug molecules to a specific point in space; or to motivate, or in other words navigate, a drug molecule or plurality of molecules along a predetermined path in space. Such motivation may take place inside the body of a person to be treated, and the drug molecule or plurality of molecules may be motivated to a predetermined point inside the person's body so that the motivated drug molecules are motivated to a predetermined location where treatment is desired. Such a location may be coincident with the location of, for example, a body organ to which it is desired to deliver the drug molecules.

### BRIEF SUMMARY OF THE INVENTION

By attaching particles with magnetic moments to a required drug, magnets located on the outside of the body generating an appropriate magnetic field may be used to steer the drug to the target location where the action of the drug is needed. However, if the drug attached to particles with magnetic moments is injected into the blood stream or intravascular a rapid dilution of the drug will occur, resulting in a significant reduction in concentration, mitigating efficacy and introducing unwanted side effects. By capturing the magnetic particles in as specific region of a magnetic field, or combination of fields, that acts like a magnetic trap, i.e., a magnetic potential well, from which the magnetic particles cannot escape given their available energy, dilution of the drug concentration is avoided. The disclosed invention implements an inventive magnetic trap with magnets on the outside of a person's body. In one embodiment, the drug is motivated to the intended location by moving the exterior magnets in a determined way, causing the magnetic trap to be motivated to the intended location inside the person's body, and therefore delivering the drug to the intended location. This intended location may be, for example, a location at which a body organ is located.

The invention is a system and method for motivating a substance, for example a drug molecule, to a predetermined location in three dimensional space by applying magnetic fields, which may be static or time-varying, to the substance. The magnetic fields may be applied by one or a plurality of magnets. In the exemplary embodiment of the invention in which the substance is a drug, for example a drug molecule or plurality of drug molecules, the magnets may be positioned outside the body of the person to be treated.

### BRIEF DESCRIPTION OF THE DRAWINGS

The accompanying drawings, which are incorporated into and form a part of the specification, illustrate one or more embodiments of the present invention and, together with the description, serve to explain the principles of the invention. The drawings are only for the purpose of illustrating the preferred embodiments of the invention and are not to be construed as limiting the invention. In the drawings:

FIG. 1 depicts a person immersed in a magnetic field, which may be a summation of a plurality of magnetic fields, and further showing a particle which may comprise a drug molecule being motivated to a predetermined location within the person's body for drug delivery to the predetermined location by the applied forces resulting from the magnetic field acting upon the particle.

### DETAILED DESCRIPTION OF THE INVENTION

Particles and any objects with magnetic moments are subjected to a force when surrounded by a non-uniform magnetic field, i.e., a magnetic field that changes with position in space. The strength of the force imposed onto the particle or object is proportional to the gradient of the magnetic field, which is measured in Tesla per meter. Depending on inertia of the particle or object, its shape, and friction caused by the surrounding medium in which the particle or object is immersed, strong gradients of 100 Tesla per meter or more may be needed to displace and motivate such particles or objects to an intended location.

Magnetic fields may be generated by currents flowing through coils made of conductive wire. The magnetic fields generated by such coil configurations fall off in intensity with distance and thereby generate magnetic fields with gradients, i.e., fields that decrease with distance from the coil. The exact functional dependence of the magnetic gradient with distance from the coil depends on the size of the coil, the direction relative to the coil, and the winding configuration, or geometry, of the coil. However, in all coil configurations the field strength decreases with distance from the coil. In order to generate high field gradients it is necessary to generate a magnetic field with sufficient strength. The following example will clarify this fact. Assume a coil configuration that generates a field of 10 Tesla directly at the winding and falls off to a very small field of 0.1 Tesla over a distance of 0.1 meter. Assuming further that the fall-off is linear, which is actually not the case for practical coil configurations, but constitutes an optimistic scenario, the resulting gradient would be:

Average gradient in first 0.1 m=10 Tesla/0.1 meter=100 Tesla/meter

It is obvious for this case that the remaining gradient at a distance greater than 0.1 m from the coil is very small, since the remaining field at the position is less or equal than 0.1 Tesla. Only if this remaining field decreases further over an extremely short distance, let's say the next 0.001 m, a sizable gradient would remain:

Average gradient between 0.1 m and 0.1001 m=0.1 Tesla/0.001 meter; or

Average gradient between 0.1 m and 0.1001 m=100 Tesla/meter

If a gradient of 100 Tesla/m is possible at such a distance, it would be limited to a very short region of space.

The above example shows that in order to generate large field gradients at large distances from a coil, the magnetic field at the coil itself has to be extremely strong.

As shown in the example, gradients of 100 Tesla/m or higher at a distance of 0.1 m from a coil over a distance of more than 0.001 m require fields of more than 10 Tesla at the coil itself. With normal conducting coils, typically made from copper conductor, such fields are only possible with large setups, requiring large amounts of cooling, typically large quantities of water passing through the conductor. Alternatively, coils comprising superconducting materials may comprise the invention, requiring electric currents of much less magnitude than normal conducting coils.

Gradient coils with gradient fields of 100 Tesla/m or more can however be generated in pulsed coils. Operating the coils in a pulsed mode can reduce the energy deposited into the coil by ohmic heating as compared to a non-pulsed mode of operation. The duty cycle in which the coil is energized may be reduced in order to reduce the total amount of energy deposited into a coil. The coil can be powered with short current pulses, which are spaced out in time to reduce the total power per time interval. Thus, an embodiment of the invention comprises pulsed coils. A pulsed coil is one in which the electrical current flowing through the magnet conductor is pulsed; i.e., is not constant over a selected period of time. The shape of the pulse may take any shape; for example rectangular, sinusoidal, Gaussian, or any other pulse shape. The following example shows the principle of reduction of energy deposited into the magnet coils when pulsed electrical current is utilized instead of a constant current:

It is assumed that a current of I=1000 A is needed to generate the required magnetic field in a coil. It is further assumed that the coil has an ohmic resistance of R=0.1 ohm. If the coil is powered continuously the power consumption is:

Power=I²*R=1000 A*1000 A*0.1 ohm=10⁵ W

The energy deposited into the coil per second therefore is:

Deposited energy=10⁵ W*1 second=10⁵ Joule

Such deposited energies can easily damage the coil by overheating. This problem is enhanced by the fact that the conductor resistivity rises with temperature and the deposited energy is even increasing, once the conductor starts to heat up.

However, if the coil is pulsed with a current pulse length of 0.001 sec and the coil is only pulsed 10 times per second, the energy deposited into the coil is reduced by a factor of a hundred:

Deposited energy=10⁵ W*10 pulses/sec*0.001 sec/pulse=10³ Joule

Thus, an embodiment of the invention uses electrical short pulses to excite the magnet coil, and by controlling the duty cycles, i.e., the number of pulses per second, the energy deposited into the coil can be reduced and overheating of the coil can be avoided.

Pulsed coil operation also facilitates the motivation of particles and objects immersed in liquids or other media. Differing liquids or other media may exhibit various values of viscosity, which in turn defines the friction between the particle and the media, or said another way, defines the resistance of the media to the motivation of the partible by the applied magnetic field. If the immersed particle is experiencing a pulsed magnetic field with a certain gradient, each pulse will transfer a certain momentum Δp onto the particle or object, which is given by integrating the time dependent force F(t) over the pulse length:

Δp=∫F(t)dt

Due to friction in the viscous liquid, the transferred momentum will be dissipated over a certain distance and the particle or object will move for a certain distance and come to a stop. By changing the direction of the gradient field and direction, the particle or object can be navigated, or motivate, through the viscous liquid in a highly controlled manner on a predetermined path, to a predetermined location.

Navigation with a constant magnetic field gradient (not pulsed) on the other hand, is more difficult to realize. It would not only require special coil designs that can sustain the high energy deposition into the coil conductor without overheating as described above, but would also require an elaborate control system for the direction of the gradient field, since the particle or object would constantly move with a velocity defined by the field gradient and the friction in the viscous liquid.

For changing the direction of the gradient field for controlled directional navigation, various schemes of pulsed coils are presented in this patent application. In one embodiment, the coil generating the gradient field is mounted on a multi-axis robotic manipulator, which allows orienting the coil as needed to achieve the desired motivation of the particle. In an alternative embodiment, a system of coils is used, either stationary or mounted individually on robotics arms, which provide the ability to adjust the gradient field direction of the superimposed field by either changing the position of individual coils and/or by adjusting the electrical current pulse amplitudes to individual coils and thereby adjusting their contribution to the vector sum of all coils. A system of coils instead of a single coil has the additional advantage of reducing the field strength requirements on the individual coils, since the total gradient then is the sum of the superimposed gradients from the individual coils.

The momentum transferred to the particle or object can be controlled with the current pulse amplitude in single coil embodiments of the invention, or the pulse amplitudes in the individual coils, in those embodiments in which a plurality of coils comprise the invention.

As described herinbefore, particles traditionally must possess a magnetic moment in order to be affected and moved by the magnetic field gradients. As an alternative, the invention comprises a system and method in which magnetic moments are induced into particles that exhibit electrical conductivity, like particles made from gold, silver or any other conductive or semi-conductive material. A pulsed magnetic field produces a magnetic flux in space that changes with time, i.e., the applied magnetic flux is increasing when the pulse is switched on and decreases when the pulse height falls to zero. A voltage Ui_{nd} is induced into the conductive particles in such a pulsed magnetic flux. The voltages thus induced are proportional to the rate of change of the magnetic flux Φ and therefore proportional to the change of electric coil current with time.

U_{ind}=−dΦ/dt∝dIₚᵤₗₛₑ/dt

The voltage induced into a conductive particle creates a current, which in turn generates a magnetic moment. This induced magnetic moment is always opposite to the inducing field under Lenz's Rule and the particle is therefore repelled. It can be seen from the above equation that the strength of the induced magnetic moment is proportional to the rise or fall time of the current pulse. As a result, also the momentum transferred to the particle is proportional to the rise and fall time of the current pulse.

If a current pulse with equal rise and fall time is being used, the particle or object acted on moves in one direction when the current pulse rises and in the opposite direction when the current pulse decreases. Under these conditions the net displacement of the particle is zero, since it is first accelerated in one direction and then back into the opposite direction by the same amount. However, using a current pulse with a short rise time and a slow fall time at the trailing edge, the particle will have a net displacement, and vice versa. The fast rising pulse edge will cause a significant momentum transfer; while the opposite momentum transfer from the trailing pulse edge is significantly reduced.

Using equal rise and fall times for the current pulses in the coils, the oscillating conductive particle can be heated by the induced currents for applications where such an effect is beneficial.

### Particle Navigation Using Magnetic Traps

As described above suspended particles with magnetic moments can be navigated in three dimensional space with pulsed magnetic gradient fields. However, for practical applications such as medical applications, not only single particles, but large assemblies of small particles need to be navigated. Brownian motion in the carrier material, for example blood, will tend to randomly distribute the particles and the extension of the particle cloud will continuously grow with a corresponding decrease in particle density. It would therefore be advantageous to have the magnetic particle cloud contained in a potential well, which counteracts the smearing and dilution of the particle cloud.

If one considers a magnetic trap, i.e. a magnetic field that is rapidly increasing in field strength starting from a central point with a field strength close to zero, only diamagnetic particles would drift towards the central point and would be captured. Paramagnetic and ferromagnetic particles would drift towards increasing field strength. Unfortunately, diamagnetic moments of all substances (except superconducting substances) are orders of magnitude smaller than the magnetic moments of paramagnetic or ferromagnetic substances, and very high magnetic fields would be required for diamagnetic particles to counteract Brownian motion or other effects that try to geometrically smear given particle distributions.

The effect on induced magnetic moments, however, is different. As mentioned above, if a current is induced into a conductive particle, due to Lenz' rule the particle is pushed in the direction of low fields. Otherwise, the induced current would actually increase when the particle moves in the direction of increasing field strength and energy conservation would be violated. Using conductive particles and a pulsed magnetic trap would therefore accumulate these particles in the field strength minimum.

Magnetic traps, i.e. configurations in which the field strength is increasing in all three spatial dimensions can be built. A magnetic field of multipole order greater than one exhibits a field strength that increase in radial direction starting from the axis of the magnet. Superimposing two opposing solenoid fields that generate fields along the axis of the magnet to the multipole field creates a central volume with very low field strength and rapidly rising field strength in all 3D spatial directions. In this respect multipole fields with the highest multipole order are best, since the fields increase in radial direction with radius to the power of n−1, where n is the multipole order. An octupole or decapole is therefore better for this application than a quadrupole. Such magnets systems even with openings for optical inspection of the central volume can be built

As in the original particle navigation system, normal conducting magnets are the best choice. As described, pulsed magnetic fields with very short rise time are required, which makes superconducting coils difficult due to AC losses, and by pulsing the coils as needed very high fields can be generated with normal conducting magnets.

By moving the magnet system that generates the magnetic trap appropriately, the captured particle cloud can be steered to the desired location. For the extremities of the human body, such magnets would be of modest size. For the main part of the body rather large magnet systems would be required.

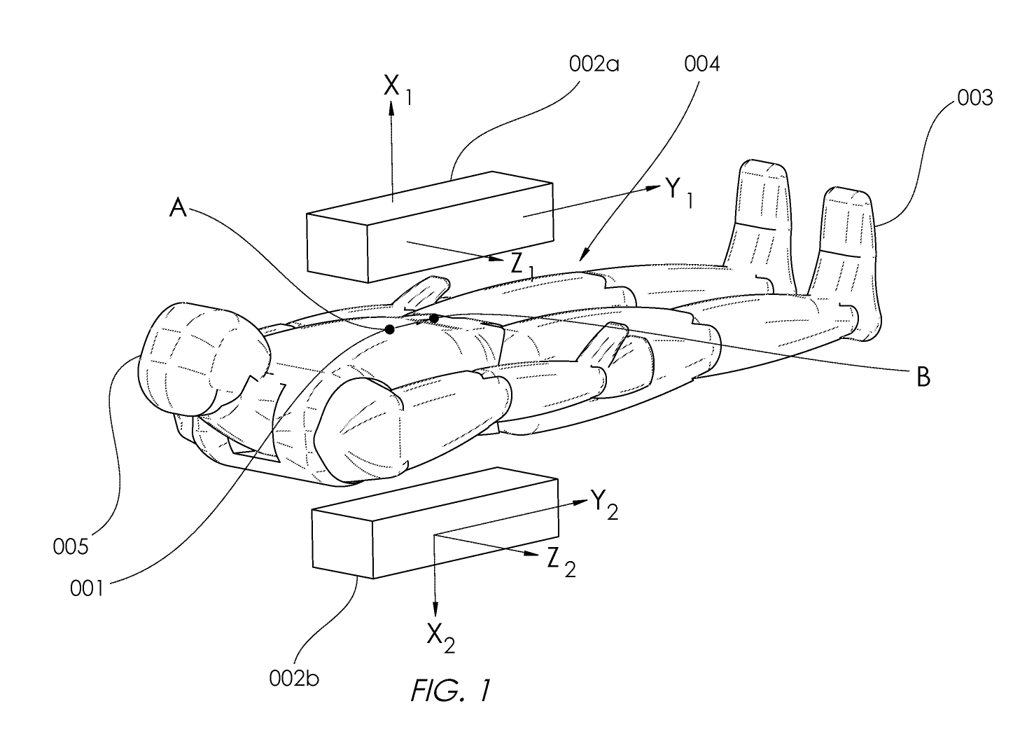

Referring to FIG. 1, a perspective view of an exemplary use of an embodiment of the invention is depicted. This specific exemplary use is that of delivering drug molecules from point A to a specific predetermined location B in a patient's body 004. Such delivery of a drug to a specific location may be beneficial, for example, for targeted treatment of specific organs. The system may comprise any number of magnets. A patient may lie between in proximity to a magnet, or between a plurality of magnets with head 005 and feet 003 oriented as shown. In the example shown, two magnets 002a and 002b are depicted. Each of magnets 002a and 002b contribute a magnetic field resulting in a superimposed magnetic field as hereinbefore described. A magnetic trap 001 may be formed inside the body of the patient at position A by the superimposition of each of the magnetic fields. The drug molecules are captured by the magnetic trap at position A, and are motivated to position B by the translation of magnetic trap 001 from position A to position B. This motivation of magnetic trap 001 is caused by the physical translation of magnets 002a and 002b along the x₁, y₁, and/or z₁ axes, and along the x₂, y₂ and/or z₂ axes, respectively. For example, each of magnets 002a and 002b may be disposed upon a three-axis positioning apparatus that is controllable for translating the magnets to a desired configuration for achieving the desired magnetic trap location. Numerical techniques may be used to determine the correct position of the magnets for achieving the desired location for the magnetic trap. Once the drug molecules captured in magnetic trap 001 have been motivated to position B, the magnetic field may be maintained in a static state, holding the drug molecules in place until they have been absorbed or otherwise utilized by the patient's body. This is but one of many uses of the invention.

Additional uses of the invention include delivering nano machines, nano particles or other substances or objects to a specific location within a patient's body.

What is claimed is:

1. A system for motivation of a particle or plurality of particles to a predetermined location, comprising:
at least one particle to be motivated to a predetermined location, said at least one particle or plurality of particles located within a medium;
said at least one particle comprising a magnetic moment;
at least one applied magnetic field of strength sufficient to motivate said at least one particle to a predetermined location in said medium.

2. The system of claim 1, wherein said at least one magnetic field forms a magnetic trap for trapping said at least one particle therein.

3. The system of claim 2, wherein said at least one applied magnetic field is comprised of a plurality of magnetic fields, and wherein said magnetic trap is a region where said plurality of magnetic fields sum to a null point forming a magnetic potential well trapping said at least one particle therein.

4. The system of claim 1 wherein said at least one particle is attached to a drug molecule; wherein said magnetic trap is formed within the body of an animal, and wherein said predetermined location is a location in the animal body at which it is desired to deliver said drug molecule.

5. The system of claim 1, wherein said applied magnetic field is generated by a plurality of magnets, the magnetic field of each magnet of said plurality of magnets superimposed, said superimposed fields forming a magnetic trap.

6. The system of claim 4 in which said animal is a human, and in which said predetermined location is a location at which it is desired to delivery said drug molecule.

7. The system of claim 6 wherein said applied magnetic field is generated by a plurality of magnets, the magnetic field of each magnet of said plurality of magnets superimposed, said superimposed fields forming a magnetic trap.

8. A system for motivation of a particle or plurality of particles to a predetermined location, comprising:
at least one particle to be motivated to a predetermined location, said at least one particle or plurality of particles located within a medium;
at least one magnet providing a time varying magnetic field;
said at least one particle immersed in said magnetic field;
said time varying magnetic field inducing a voltage in said particle, causing said particle to be motivated in a desired direction, such that said particle is motivated to a predetermined location.

9. The system of claim 8, wherein said time varying magnetic field forms a magnetic trap for trapping said at least one particle therein.

10. The system of claim 9, wherein said time varying applied magnetic field is comprised of a summation of a plurality of time varying magnetic fields, and wherein said magnetic trap is a region where said plurality of magnetic fields sum to a null point forming a magnetic potential well trapping said at least one particle therein.

11. The system of claim 8 wherein said at least one particle comprises a drug molecule; wherein said magnetic trap is formed within the body of an animal, and wherein said predetermined location is a location in the animal body at which it is desired to deliver said drug molecule.

12. The system of claim 8, wherein said applied magnetic field is formed by a plurality of magnets, the magnetic field of each magnet of said plurality of magnets superimposed, said superimposed magnetic fields forming a magnetic trap.

13. The system of claim 11 in which said animal is a human, and in which said predetermined location is a location at which it is desired to delivery said drug molecule.

14. The system of claim 13 wherein said applied magnetic field is generated by a plurality of magnets, the magnetic field of each magnet of said plurality of magnets superimposed, said superimposed fields forming a magnetic trap.

15. The system of claim 8, wherein said time varying magnetic field is further defined as comprising a series of pulses of magnetic field intensity, each pulse having a leading edge comprising a period of increasing magnetic field intensity, and a trailing edge comprising a period of decreasing magnetic field intensity.

16. The system of claim 9, wherein said time varying magnetic field is further defined as comprising a series of pulses of magnetic field intensity, each pulse having a leading edge comprising a period of increasing magnetic field intensity, and a trailing edge comprising a period of decreasing magnetic field intensity.

17. The system of claim 10, wherein said time varying magnetic field is further defined as comprising a series of pulses of magnetic field intensity, each pulse having a leading edge comprising a period of increasing magnetic field intensity, and a trailing edge comprising a period of decreasing magnetic field intensity.

18. The system of claim 11, wherein said time varying magnetic field is further defined as comprising a series of pulses of magnetic field intensity, each pulse having a leading edge comprising a period of increasing magnetic field intensity, and a trailing edge comprising a period of decreasing magnetic field intensity.

19. The system of claim 13, wherein said time varying magnetic field is further defined as comprising a series of pulses of magnetic field intensity, each pulse having a leading edge comprising a period of increasing magnetic field intensity, and a trailing edge comprising a period of decreasing magnetic field intensity.

20. The system of claim 14, wherein said time varying magnetic field is further defined as comprising a series of pulses of magnetic field intensity, each pulse having a leading edge comprising a period of increasing magnetic field intensity, and a trailing edge comprising a period of decreasing magnetic field intensity.

21. The system of claim 15, wherein said time varying magnetic field is further defined as comprising a series of pulses of magnetic field intensity, each pulse having a leading edge comprising a period of increasing magnetic field intensity, and a trailing edge comprising a period of decreasing magnetic field intensity.

22. The system of claim 15, wherein said period of decreasing magnetic field intensity is greater than said period of increasing field intensity.

23. The system of claim 15 wherein said magnetic field is a multipole magnetic field of order greater than one.

24. The system of claim 15 wherein said magnetic field is an octopole magnetic field.

25. The system of claim 15 wherein said magnetic field is a decapole magnetic field.

26. The system of claim 15 wherein said particle is a diamagnetic particle.

27. A method for motivating a particle to a desired location using at least one magnetic field, comprising the steps of:
providing a magnetic field comprising a magnetic trap, said magnetic field generated by at least one magnet:
providing a particle with a magnetic moment immersed in said magnetic field;
manipulating said at least one magnet such that said magnetic trap is motivated to a desired location.

28. The method of claim 27, wherein the step of providing a magnetic field is further defined as providing a magnetic field comprising a summation of a plurality of magnetic fields.

29. The method of claim 28, wherein the step of providing a plurality of magnetic fields is further defined as each magnetic field of said plurality of magnetic fields is provided by a separate, individual magnet.

30. The method of claim 27, wherein the step of providing a particle is further defined as providing a particle comprising a drug molecule, and wherein the step of manipulating said at least one magnet such that said magnetic trap is motivated to a desired location is further defined in that said desired location is defined to be a location in an animal body at which it is desired to deliver said drug molecule.

31. The method of claim 28, wherein the step of providing a particle is further defined as providing a particle comprising a drug molecule, and wherein the step of manipulating said at least one magnet such that said magnetic trap is motivated to a desired location is further defined in that said desired location is defined to be a location in an animal body at which it is desired to deliver said drug molecule.

32. The method of claim 29, wherein the step of providing a particle is further defined as providing a particle comprising a drug molecule, and wherein the step of manipulating said at least one magnet such that said magnetic trap is motivated to a desired location is further defined as said desired location is defined to be a location in an animal body at which it is desired to deliver said drug molecule.

33. A method for motivating a particle to a desired location using at least one magnetic field, comprising the steps of:
providing at least one particle to be motivated to a predetermined location, said at least one particle or plurality of particles located within a medium; and
providing at least one magnet providing a time varying magnetic field; wherein
said at least one particle is immersed in said magnetic field; and wherein
said time varying magnetic field induces a voltage in said particle, causing said particle to be motivated in a desired direction, such that said particle is motivated to a predetermined location.

34. The method of claim 33, wherein said magnetic field is further defined as providing a magnetic field comprising a summation of a plurality of magnetic fields.

35. The method of claim 34, wherein each magnetic field of said plurality of magnetic fields is provided by a separate, individual magnet.

36. The method of claim 33, wherein said at least one particle is further defined as being at least one drug molecule, and wherein the step of manipulating said at least one magnet such that said magnetic trap is motivated to a desired location is further defined in that said desired location is defined to be a location in an animal body at which it is desired to deliver said drug molecule.

37. The method of claim 33, wherein said at least one particle is further defined as being at least one drug molecule, and wherein the step of manipulating said at least one magnet such that said magnetic trap is motivated to a desired location is further defined in that said desired location is defined to be a location in an animal body at which it is desired to deliver said drug molecule.

38. The method of claim 35, wherein said at least one particle is further defined as at least one drug molecule, and wherein the step of manipulating said at least one magnet such that said magnetic trap is motivated to a desired location is further defined as said desired location is defined to be a location in an animal body at which it is desired to deliver said drug molecule.

39. The method of claim 33, in which said time-varying magnetic field is produced by a time-varying electrical pulse in coils comprising each of said magnets.

40. The method of claim 33, in which said time-varying magnetic field is produced by a time-varying electrical pulse in coils comprising each of said magnets, each pulse having a leading edge causing a period of increasing magnetic field intensity, and a trailing edge causing a period of decreasing magnetic field intensity, and wherein said trailing edge period is greater than said leading edge period.
